(19) United States
(12) Patent Application Publication
(10) Pub. No.: US 2005/0101888 A1
(43) Pub. Date: May 12, 2005

(54) Therapeutic spinal rest and support

(76) Inventor: Ryan F. West, Santa Cruz, CA (US)

Correspondence Address: MICHAEL A. GUTH, 2-2905 EAST CLIFF DRIVE, SANTA CRUZ, CA, 95062, US

(21) Appl. No.: 10/706,165

(22) Filed: Nov. 12, 2003

(51) Int. Cl. A61F 7/02; A61F 7/08

(52) U.S. Cl. 601/15; 607/108; 607/112; 607/114

(57) ABSTRACT

A spinal rest adapted to provide either lumbar or thoracic support or a combination of the two. The spinal rest and support allows for the insertion of thermal packs such that hot or cold may be applied to the user. The thermal packs reside in an indentation within the front surface of the back support. The back rest provides support for proper curvature of the spine in the thoracic and lumbar region, while also able to support a thermal pack.

### BACKGROUND

1. Field of the Invention

This invention relates to a therapeutic spinal rest device.

2. Description of the Related Art

Proper support for the back is important to persons with back problems. A back support may be part of a therapeutic program for one with back problems, or in general for persons seeking to avoid future back problems. In addition to proper back support, a preventative or therapeutic program may involve the application of heat or cold to the back.

Some back supports are fashioned conform completely to a person's shape in the region to be supported while pinned between that person and the backrest of a seat. An example of such a device is U.S. Pat. No. 5,948,013 to Swezey et. al. With this device, the support is placed in the space between the person's back and the backrest of a seat, and then inflated to conform the space. A disadvantage of this approach is that sometimes support is desired to be provided in a different fashion. In some instances, it is preferred that the support be more rigid such that the person's supported area conform to the back support and not vice versa, or at least some combination of both cases.

U.S. Pat. No. 5,948,013 also discloses the placement of a thermal packet in front of the back support between the back support and the user's back. A disadvantage of this method is that the different rigidity and/or compressibility of the thermal packet may affect the way in which the user's back is supported, transferring pressure primarily to the area of the packet and away from other areas of the back support.

The superficial heating and cooling modalities used in sports medicine setting are classified as infrared modalities. Heating modalities are referred to as thermotherapy. The use of cold is referred to as cryotherapy. Perhaps the most effective use of the infrared modalities is for analgesia, reducing the sensation of pain associated with injury. The infrared modalities primarily stimulate the cutaneous nerve receptors. Within the philosophy of an aggressive program of rehabilitation the reduction of pain as a means of facilitating therapeutic exercise is a common practice.

What is called for is a spinal rest and support that supports the lumbar, thoracic, or other regions and can also accommodate the insertion of thermal packets for thermal treatment of the user's back while maintaining more even pressure across the supported area. What is also called for is a spinal rest and support that can be made from a less resilient material, if desired for a particular user, such that the user's back is supported into a slightly different position than if no back support were present. Such a device is highly beneficial because it allows the spine to rest and also provides support for proper curvature of the spine. With the addition of hot and/or cold packs, the spinal rest will increase muscle relaxation, release tension, and decrease nerve agitation while sitting or while driving a motor vehicle.

### SUMMARY

A spinal rest and support adapted to provide either lumbar or thoracic support or a combination of the two for proper curvature of the spine. The back support allows for the insertion of thermal packs such that hot or cold may be applied to the user. The thermal packs reside in an indentation within the front surface of the back support.

### BRIEF DESCRIPTION OF THE DRAWINGS

FIG. 1 is a perspective view of a first embodiment of the present invention.

FIG. 2 is another perspective view of a first embodiment of the present invention.

FIG. 3 is a perspective view of a second embodiment of the present invention.

FIG. 4 is a top view of a second embodiment of the present invention.

FIG. 5 is a side view of a second embodiment of the present invention.

FIG. 6 is a perspective view of a third embodiment of the present invention.

FIG. 7 is a top view of a third embodiment of the present invention.

FIG. 8 is a perspective view of a thermal pack.

FIG. 9 is a side view of a thermal pack.

FIG. 10 is a perspective view of a one embodiment of the present invention including a cover.

FIG. 11 is cutaway view of one embodiment of the present invention including a cover.

FIG. 12 is a perspective view of a thermal pack including a cover.

FIG. 13 is a perspective view of one embodiment of the present invention.

FIG. 14 illustrates the three segments of the mobile spinal column.

FIG. 15 is a side view of a person sitting.

FIG. 16 is a side view of a person sitting utilizing one embodiment of the present invention.

FIG. 17 is a side view of a person sitting utilizing one embodiment of the present invention.

FIG. 18 is a side view of a person sitting utilizing one embodiment of the present invention.

FIG. 19 is an illustration of an embodiment of the present invention utilizing another embodiment of the attachment portion.

### DETAILED DESCRIPTION

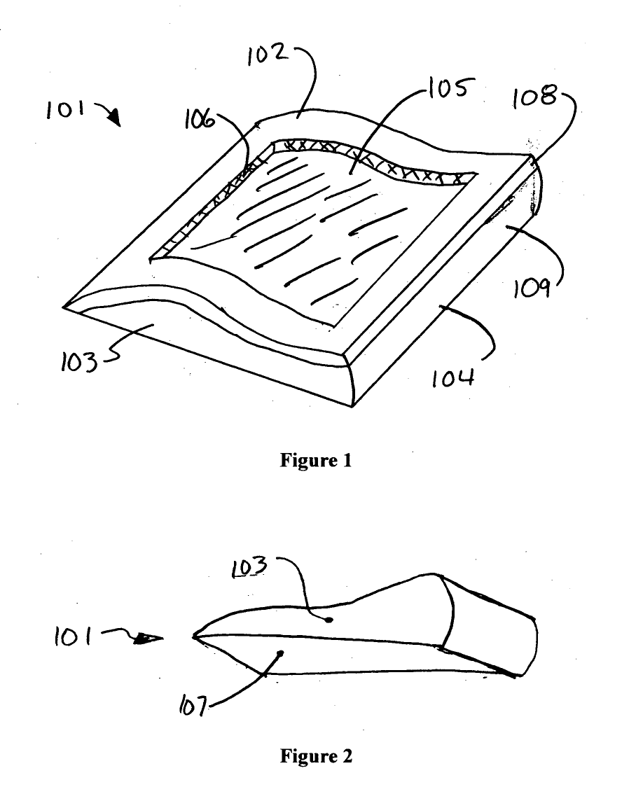

In one embodiment of the present invention, as seen in FIGS. 1 and 2, spinal rest main body portion 101 has a front surface 102. An indentation 105 in the front surface 102 of back support main body portion 101 allows for the placement within indentation 105 of a thermal pack. The indentation periphery 106 of indentation 105 lies within the boundaries of the front surface 102 in this embodiment. In other embodiments, indentation 105 may continue to one or more edges of front surface 102.

Bottom surface 104 may be flat or curving along one of its planar directions. Although the end of front surface 102 opposite that which intersects with bottom surface 104 meets with back surface 107 in this embodiment, front surface 102 may meet with the back panel at all edges in some embodiments.

As shown in FIG. 1, spinal rest main body portion 101 may be comprised of two layers. Top layer 108 sits over main layer 109 in this embodiment. Top layer 108 may fully contain indentation 105, allowing for the manufacture of spinal rest main body portion 101 by the joining of two layers. In other embodiments, more layers may be used. Layers may be used to manipulate the stiffness of back support main body portion 101.

In one embodiment of the present invention, as seen in FIGS. 1 and 2, side panel 103 has a contour adapted to provide therapeutic back support. Pad back 107 is adapted to be placed against the back rest of a chair, car seat, or other similar item. Front surface 102 is contoured in the same fashion as side panel 103 in this embodiment. Front surface 102 may have a different profile at different points across its surface in some embodiments.

Side panel 103 has a profile indicative of a profile which would be used for lumbar support.

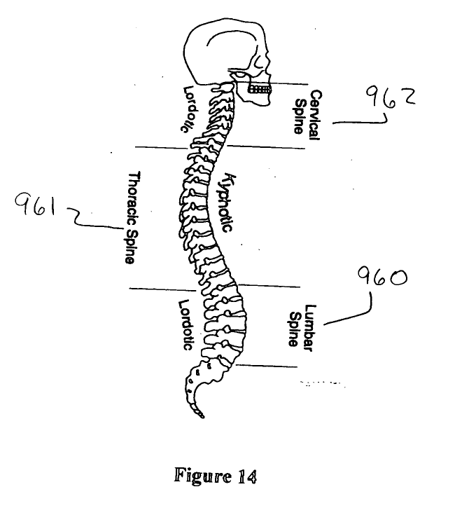

FIG. 14 illustrates the three segments of the mobile spinal column with their normal curvature. Lumbar spine 960 is the bottom most portion of the spinal column. Thoracic spine 961 is the middle portion of the spinal column above the lumbar spine 960. Cervical spine 962 is the upper portion of the spinal column above the thoracic spine 961.

Indentation 105 may be sized to accommodate available thermal packs. Some thermal packs are 8.5 inches by 8.5 inches and approximately 0.75 inches thick. Indentation 105 may be sized to be 9.5 inches by 9.5 inches by 0.75 inches deep in some embodiments. Indentation 105 allows for the placement of a thermal pack into back support main body portion 101 without allowing the shape and rigidity of the thermal pack to dictate the front surface profile of the back support when in use. The compressibility of the thermal pack may be different than the compressibility of the material of the back support. In such cases, the overall compressibility may still be dictated by the back support in some embodiments.

Spinal rest main body portion 101 may be made from one piece of material or multiple pieces. Spinal rest main body portion 101 may be constructed from urethane foam, polyurethane foam, or other suitable materials. Spinal rest main body portion 101 may be constructed of open cell foam, closed cell foam, or combinations of these and other materials.

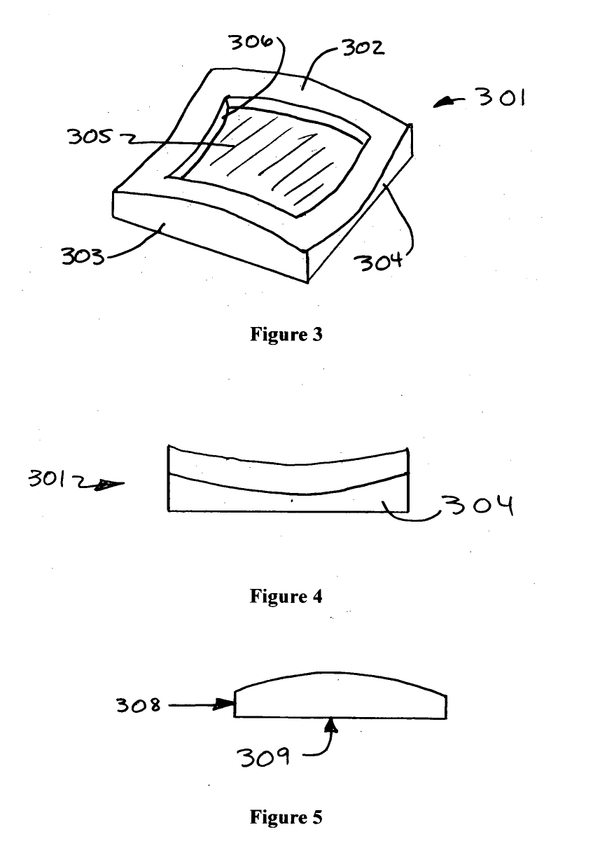

In another embodiment of the present invention, as seen in FIG. 3, spinal rest main body portion 301 has front surface 302 which is convex in one planar direction and concave in another planar direction. Side panel 303 has a contour adapted to the purpose of providing thoracic and lumbar support. Bottom panel 304 is of similar or the same profile contour as top panel 308 in this embodiment. In other embodiments, the bottom panel and the top panel may be of different profile contours. Front surface 302 has an indentation 305. Indentation 305 is adapted to allow for the placement of a thermal pack. Alternatively, a pad may be placed into indentation 305. Indentation 305 is surrounded by an indentation periphery 306.

Side panel 303 has a profile contoured to provide back support. Back surface 309 is adapted to be placed against a back of a chair, car seat, or other similar item. As seen in FIG. 5, back surface 309 in this embodiment. The back surface may be flat or may be contoured in some embodiments.

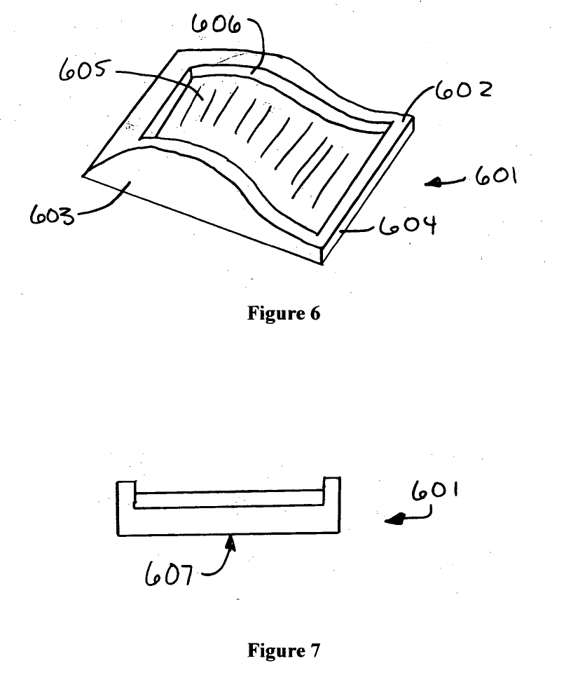

In another embodiment of the present invention, as seen in FIGS. 6 and 7, spinal rest main body portion 601 is shaped to provide support for proper curvature of the lumbar-thoracic region. Side panel 603 has a profile contour that is representative of the profile across front surface 602 in this embodiment.

Indentation 605 allows for the placement of a thermal pack, which then fills in indentation 605 and allows for a consistent front surface layer across back support main body portion 601. Indentation periphery 606 outlines indentation 605. In some embodiments, the top of the thermal pack may be above or below the front surface 602. Bottom surface 604 and back surface 607 are flat and planar in this embodiment.

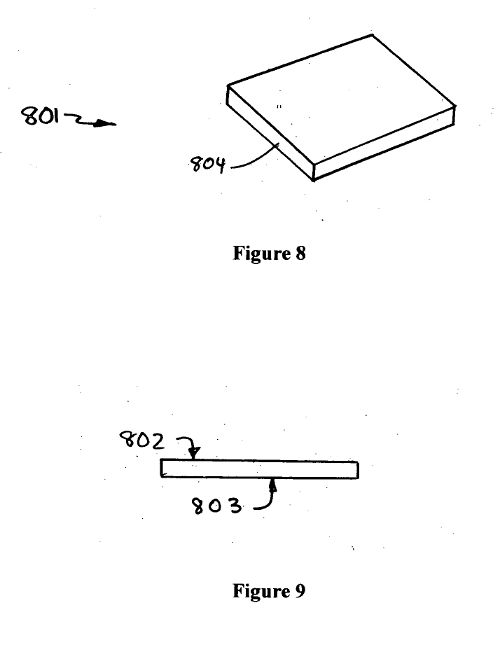

As seen in FIGS. 8 and 9, thermal pack 801 is sized to be fit into the indentations in the back support main body portion indentations. Thermal pack periphery 804 will fit within the indentation periphery when the thermal pack is placed into the main body portion. The thermal pack may consist of thermal gel encased in a container such as a plastic, or may of other suitable design including currently available hot and cold packs. Alternatively, the thermal pack 801 may be substituted with a pad to fill in the main body portion indentation when no thermal effect is desired.

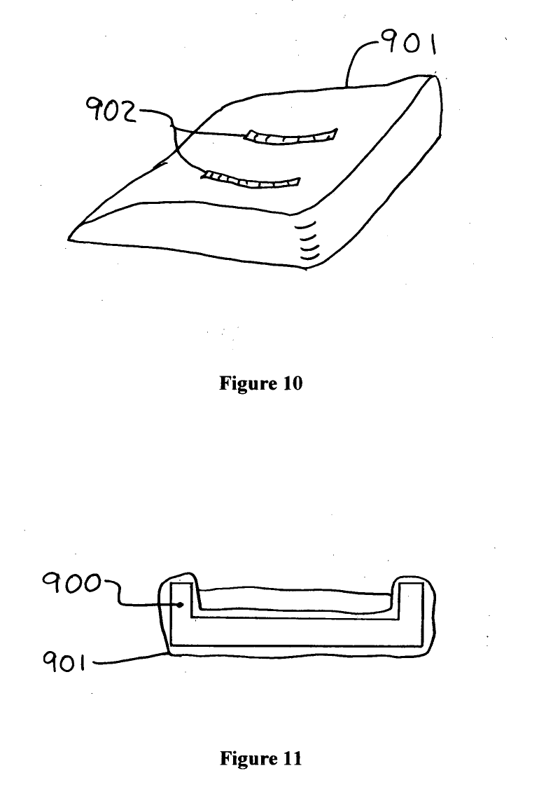

In some embodiments of the present invention, as seen in FIGS. 10 and 11, the spinal rest main body 900 is covered with a main body cover 901. The main body cover 901 may be made out of cloth or other appropriate material. Main body cover 901 is adapted to substantially conform to the outside surface of back support main body 900. Main body cover 901 may be slipped over back support main body 900, or may be adhered in another fashion. Attachment portions 902 are on the outside of main body cover 901 in this embodiment. Attachment portions 902 may be made of Velcro type fasteners or other appropriate means. Attachment portions 902 are in the area of the indentation in the main body in this embodiment.

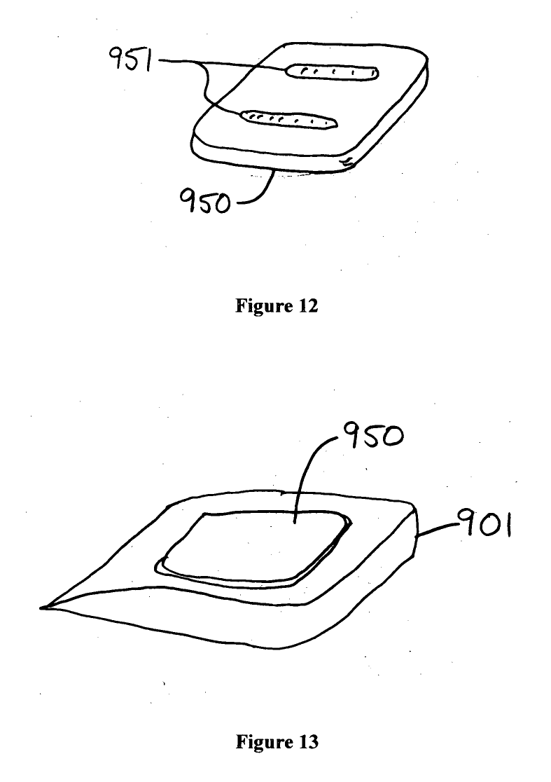

In some embodiments of the present invention, as seen in FIG. 12, a thermal pack is covered with a thermal pack cover 950. Thermal pack cover 950 may be made of cloth or other appropriate material. Attachment portions 951 are adapted to removably fasten thermal pack cover 950 to the main body cover 902. As seen in FIG. 13, the covered thermal pack is placed into and removably fastened to the main body cover 901.

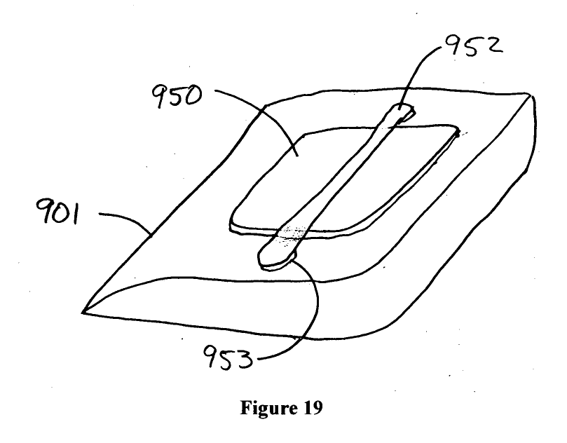

FIG. 19 illustrates another embodiment of the present invention wherein the covered thermal pack is attached to the main body using an alternate method. The thermal pack cover 950 is shown covering the thermal pack and residing in the indentation of main body. The main body is covered by the main body cover 901. Attachment strap 952 retains the covered thermal pack into the indentation of the main body. Strap retention portion 953 is attached to the main body cover 901. Attachment strap 952 attaches to the strap retention portion 953 and in so doing retains the thermal pack in the indentation of the main body. In some embodiments, the attachment strap may go completely around the main body. In some embodiments, the attachment strap may be fastened to the main body cover 901 using a more permanent method such as sewing. In some embodiments, the attachment strap 952 may be made partially or wholly of elastic.

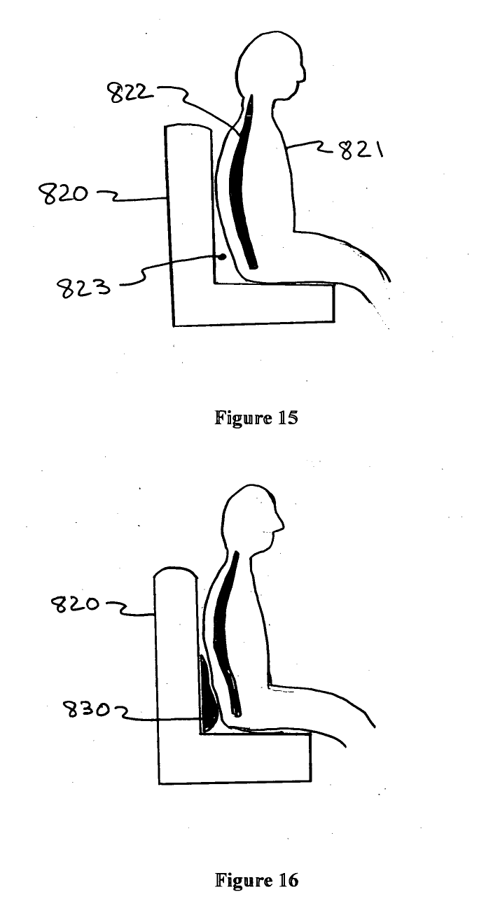

FIG. 15 illustrates a person 821 sitting in a chair or other seating device 820 sits with a curvature of the spine 822. Sitting with a void 823 behind the spine 822 makes the spine rounded. This position may stretch the thoracolumbar fascia and the lumbar plexus. This may increase pain and nerve tension.

FIG. 16 illustrates one use of one embodiment of spinal rest 830 of the present invention. Used in this fashion, spinal rest 830 does not allow for such curvature of the lumbar and thoracic spine as seen in FIG. 15. This reduces nerve tension and stretching of the spine and fascia.

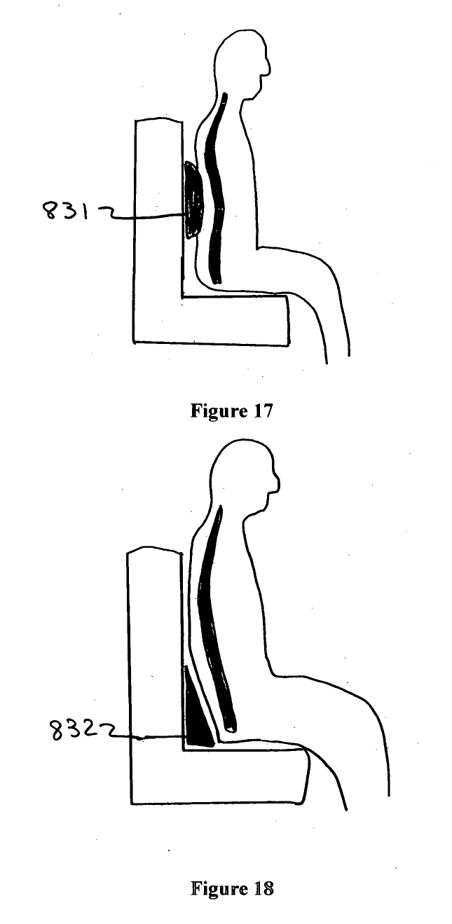

FIGS. 17 and 18 illustrate uses of other embodiments of the spinal rest 831, 832. Although the spinal rest is shown here used when a person is in a sitting position, a person may be is other positions, such as lying down, when utilizing this invention.

In some embodiments of the present invention, the spinal rest is made from polyurethane foam. There are a number of physical properties of flexible polyurethane foam that can be used when selecting foam for different applications. Two of the important properties are density and indentation force deflection (IFD). The density is a measurement of mass per unit volume. Density affects foam durability and support. IFD is a measurement of foam firmness. IFD is measured by indenting a foam sample 25% of its original height. Different combinations of density and IFD may be used depending upon the desired result in different embodiments of the invention. Densities for use in cushioning applications may range from 0.5 to 3.0 pcf. IFD may range from 10-50 pounds.

As evident from the above description, a wide variety of spinal rests and supports may be configured from the description given herein and additional advantages and modifications will readily occur to those skilled in the art. The invention in its broader aspects is, therefore, not limited to the specific details, representative apparatus and illustrative examples shown and described. Accordingly, departures from such details may be made without departing from the spirit or scope of the applicant's general inventive concept.

1. A spinal rest comprising:
a main body portion, said main body portion comprising:
one or more layers of materials;
a front surface;
a back surface;
wherein said front surface has a indentation in said front surface.

2. The spinal rest of claim 1 wherein said main body portion further comprises a bottom surface.

3. The spinal rest of claim 1 wherein said indentation resides fully within the periphery of said front surface.

4. The spinal rest of claim 1, wherein said indentation is adapted to allow for the placement of a thermal package substantially therein.

5. The spinal rest of claim 1, wherein said front surface is contoured to provide lumbar support.

6. The spinal rest of claim 1, wherein said front surface is contoured to provide thoracic support.

7. The spinal rest of claim 5, wherein said back surface is adapted to be placed substantially in contact with a seat backrest.

8. The spinal rest of claim 6, wherein said back surface is adapted to be placed substantially in contact with a seat backrest.

9. The spinal rest of claim 4 wherein said thermal package is substantially rectangular.

10. The back rest of claim 4 further comprising a thermal package.

11. The spinal rest of claim 1 wherein said main body portion comprises a first layer and a second layer, wherein the boundary between said first layer and second layer is relatively parallel to said first surface.

12. The spinal rest of claim 11 wherein one of said first layer and said second layer is primarily open cell foam.

13. The spinal rest of claim 5, wherein said indentation is adapted to allow for the placement of a thermal package substantially therein.

14. The spinal rest of claim 1, wherein said indentation is adapted to allow for the placement of a thermal package substantially therein.

15. The spinal rest of claim 4 further comprising a first cover, said first cover substantially in the shape of the outside surfaces of said main body.

16. The spinal rest of claim 15 wherein said first cover substantially covers said outside surfaces of said main body.

17. The spinal rest of claim 16, further comprising a second cover, said second cover adapted to cover a thermal unit, said second cover sized to fit substantially within said indentation.

18. The spinal rest of claim 15 wherein said first cover comprises cloth.

19. The spinal rest of claim 16 wherein said first cover comprises cloth.

20. The spinal rest of claim 17 further comprising a thermal unit, wherein said thermal unit is substantially within said second cover.

21. The spinal rest of claim 16 further comprising a first attachment portion fastened to the exterior of said first cover, wherein said first attachment portion is fastened to the exterior of said first cover substantially in the region wherein said first cover overlies said indentation.

22. The spinal rest of claim 17 further comprising:
a first attachment portion fastened to the exterior of said first cover, wherein said first attachment portion is fastened to the exterior of said first cover substantially in the region wherein said first cover overlies said indentation; and
a second attachment portion fastened to the exterior of said second cover, said second portion fastened to the exterior of said second cover, said first attachment portion and said second attachment portion disposed such that said first attachment portion and said second attachment portion fasten to each other while positioning said second cover substantially in the region wherein said first cover overlies said indentation.

23. The spinal rest of claim 1 wherein said indentation is approximately 9.5 inches square and approximately 0.75 inches deep.
